(19) United States
(12) Plant Patent Application Publication
(10) Pub. No.: US 2003/0106115 P1
(43) Pub. Date: Jun. 5, 2003

(54) Hybrid tea rose plant named 'Bronze Sunset'

(76) Inventor: James W. Coiner, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/016,071

(22) Filed: Dec. 5, 2001

(51) Int. Cl. A01H 5/00

(52) U.S. Cl. PLT/135

(57) ABSTRACT

A new and distinct Hybrid Tea Rose plant variety with orange flowers.

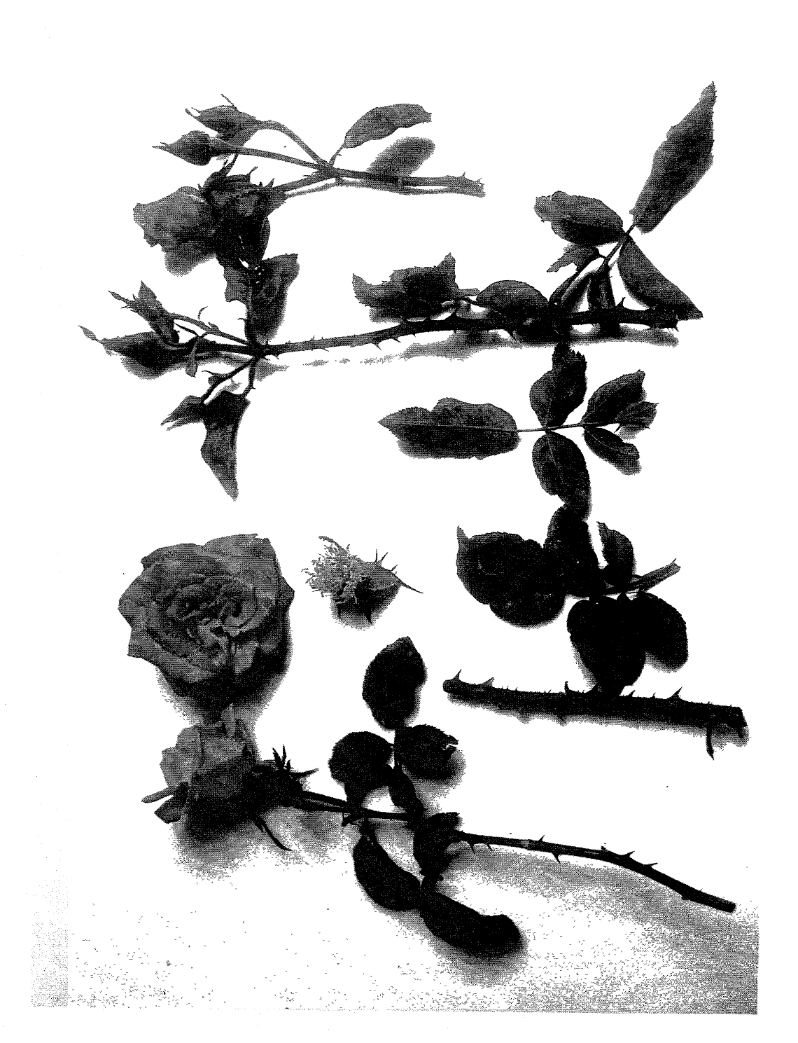

### BACKGROUND OF THE INVENTION

The present invention relates to a new and distinct variety of rose plant of the hybrid tea rose which was created by crossing as seed and pollen parents two (2) unknown rose varieties. The varietal denomination of the new variety is ‘BRONZE SUNSET’.

### SUMMARY OF THE INVENTION

Among the novel characteristics possessed by this new variety which distinguishes it from other varieties of which I am aware, is its orange flowers.

Asexual reproduction by propagation by budding of the new variety as performed in Wasco, Calif. shows that the foregoing and other distinguishing characteristics come true to form and are established and transmitted through succeeding propagations.

### BRIEF DESCRIPTION OF ILLUSTRATION

The accompanying illustration shows typical specimens of the flowers of the new variety depicted in color as nearly true as it is reasonably possible to make the same in a color illustration of this character.

### DESCRIPTION OF THE NEW VARIETY

The following is a detailed description of 2-year budded plants of the new variety , with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of specimens grown outdoors in Wasco, Calif., during November.

### PLANT

Form: Spreading

Growth: Vigorous; sprawling

Size: Height about 52 inches; width about 48 inches.

Foliage

Size.—Length about 2 ½ inches; width about 1 9/16 inches

Quantity.—About 5.

Color.—New foliage: Upper side near 137B; under side near 148A Mature foliage: Upper side near 139A; under side near 137B.

Texture.—Upper side: Smooth Under side: Matte

Edge.—Serrated.

Petioles: Top surface color near 138C; Under side near 138C

Stipules: Medium length

Auricle: Light red brown near 59C fading to green near 137B

Rachis: Few prickles

Variegation: Slight

### WOOD

New shoots

Bark.—Smooth.

Wood.—Near 59C.

Old wood

Bark.—Lots of prickles.

Wood.—Near 137D.

Flowering stem: Length about 5 ½ inches

Winter Hardiness: Completely winter hardy

Resistance to Disease: Fair

Temperature range: Between 26° and 120° F.

Internode spacing: Irregular

Preferred growing conditions: Likes full sun

Growth habit: Vigorous

Leaves per stem: About 5

### FLOWER/INFLORESCENCE

Blooming Habit: Recurrent

Bud.—About 4 ¼ inches. Form: Triangular Color: Before sepals divide color near 169A; when sepals first divide color near 170B; when petals begin to unfurl color near 41A

Sepals.—Regular. Size: Length in relation to bud, about 1 ¼ inches Color Topside near 164B; bottom near 137C

Peduncle.—Length: About 2 ½ inches Strength: Strong Color: Near 148C

Flower

Size.—About 4 ¼ inches.

Borne.—Single.

Form.—Relative good form; high centered; unfurls to loose ruffled at maturity.

Petalage: About 25

Petaloids in center.—None.

Persistence.—Normal.

Fragrance: Slight

Lasting Quality: About 2 weeks on plant; about 6 days after cutting.

Color

During first 2 days.—Inner petals at flower center topside near 41A; bottom near 19A; outer petals topside near 43A, bottom near 16C

Base of petals (Point of attachment): Near 17B

General tonality from a distance: Bronze

Color Change: Slightly darkens; remains the same after 3 or more days

Petals

Texture.—Thick/rigid.

Shape.—Wide at top, tapered to narrow bottom.

Arrangement.—Loose around pistils.

Appearance.—Satiny.

### REPRODUCTIVE ORGANS

Arrangement of stamens, Near 163B; regularly arranged

Filaments and Anthers: around styles

Color: Filaments near 162C; Anthers near 161C

Pollen: Near 168A

Styles: Bunched, uneven length

Stigmas: Normal; color near 16C

Hips/Seed Vessels/Ovaries

Shape.—Oval.

Size.—About 1/2 inch.

Color.—Near 143B.

Seeds: No seeds

I claim:

1. A new and distinct Hybrid Tea Rose plant of the variety substantially as shown and described.
